(19) United States
(12) Patent Application Publication
(10) Pub. No.: US 2004/0082916 A1
(43) Pub. Date: Apr. 29, 2004

(54) Catheter support system

(76) Inventor: Jane A. Jenkins, Clayton, OH (US)

Correspondence Address: Michael S. Neustel, Suite No. 4, 2534 South University Drive, Fargo, ND, 58103, US

(21) Appl. No.: 10/284,556

(22) Filed: Oct. 29, 2002

(51) Int. Cl. A61M 5/32

(52) U.S. Cl. 604/174

(57) ABSTRACT

A catheter support system for effectively supporting a central venous catheter and for providing tactile feedback to a patient. The catheter support system includes a support loop positionable about the neck of a patient, a connector member for fluidly connecting a catheter tube with a supply tube or an end cap, and an eyelet member attached to the connector member for receiving the support loop. If a force is applied to either the distal end of the catheter tube or at any location along the supply tube, the support loop is pulled against the neck of the patient for both supporting the catheter tubes and warning the patient of the force being applied thereto.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to central venous catheter devices and more specifically it relates to a catheter support system for effectively supporting a central venous catheter and for providing tactile feedback to a patient.

2. Description of the Related Art

Central venous catheters have been in use for years. Central venous catheterization involves the insertion of an indwelling catheter into a central vein for administering fluid and medications or for measuring central venous pressure. The catheter tubes are often times secured to the skin of the patient by utilizing conventional medical tape. Additional securing methods include looping the catheter tube and securing with medical tape, or wrapping a piece of medical tape to the distal end of the catheter tube and securing to clothing of the patient with a safety pin. The most common sites of insertion are the jugular and subclavian veins. Connectors are often times utilized at the catheter hub for allowing connection of either a cap member or a supply tube. Long-term catheter tubes are tunneled under the skin and sutured in place of the insertion site.

The main problem with conventional central venous catheterization securing systems is that they do not adequately support the catheter tube. A further problem is that catheter tubes are susceptible for being accidentally engaged causing central venous catheter dislodgement and possibly infection. A further problem is that conventional securing methods do not provide a sufficient warning to the patient when the catheter tube is being pulled upon.

While these devices may be suitable for the particular purpose to which they address, they are not as suitable for effectively supporting a central venous catheter and for providing tactile feedback to a patient. Conventional central venous catheter securing devices and systems do not provide adequate support nor do they provide adequate warning to a patient when forces are being exerted upon the catheter tube.

In these respects, the catheter support system according to the present invention substantially departs from the conventional concepts and designs of the prior art, and in so doing provides an apparatus primarily developed for the purpose of effectively supporting a central venous catheter and for providing tactile feedback to a patient.

### BRIEF SUMMARY OF THE INVENTION

In view of the foregoing disadvantages inherent in the known types of central venous catheter devices and securing methods now present in the prior art, the present invention provides a new catheter support system construction wherein the same can be utilized for effectively supporting a central venous catheter and for providing tactile feedback to a patient.

The general purpose of the present invention, which will be described subsequently in greater detail, is to provide a new catheter support system that has many of the advantages of the securing methods mentioned heretofore and many novel features that result in a new catheter support system which is not anticipated, rendered obvious, suggested, or even implied by any of the prior art securing methods, either alone or in any combination thereof.

To attain this, the present invention generally comprises a support loop positionable about the neck of a patient, a connector member for fluidly connecting catheter tubes, and an eyelet member attached to the connector member for receiving the support loop. If a force is applied to either the catheter hub or supply tube, the support loop is pulled against the neck of the patient for both supporting the catheter tube and warning the patient of the force being applied thereto.

There has thus been outlined, rather broadly, the more important features of the invention in order that the detailed description thereof may be better understood, and in order that the present contribution to the art may be better appreciated. There are additional features of the invention that will be described hereinafter and that will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a catheter support system that will overcome the shortcomings of the prior art devices.

A second object is to provide a catheter support system for effectively supporting a central venous catheter and for providing tactile feedback to a patient.

Another object is to provide a catheter support system that may be attached to patients of various sizes.

An additional object is to provide a catheter support system that can be easily attached to a patient without requiring the usage of medical tape.

A further object is to provide a catheter support system that prevents dislodgement of the central venous catheter.

Another object is to provide a catheter support system that is adjustable.

Another object is to provide a catheter support system that provides comfort, peace of mind and enhanced body image to the patient.

A further object is to provide a catheter support system that utilizes a connector member that fits various types of central venous catheters.

Another object is to provide a catheter support system that will function properly when wet or when skin oil accumulates.

Other objects and advantages of the present invention will become obvious to the reader and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the prior art illustrating the central venous catheter secured with a piece of tape with the catheter tubes hanging from thereof without support.

FIG. 2 is an upper perspective view of the present invention secured upon the patient and to the central venous catheter.

FIG. 3 is an upper perspective view of the present invention with the catheter tube being pulled upon thereby causing the support loop to pull upon the neck of the patient to immediately indicate the force.

FIG. 4 is an upper perspective view of the present invention with the catheter tube and at the supply tube removed from the connector member.

FIG. 5 is an upper perspective view of the present invention with the catheter tube and supply tube secured within the connector member.

### DETAILED DESCRIPTION OF THE INVENTION

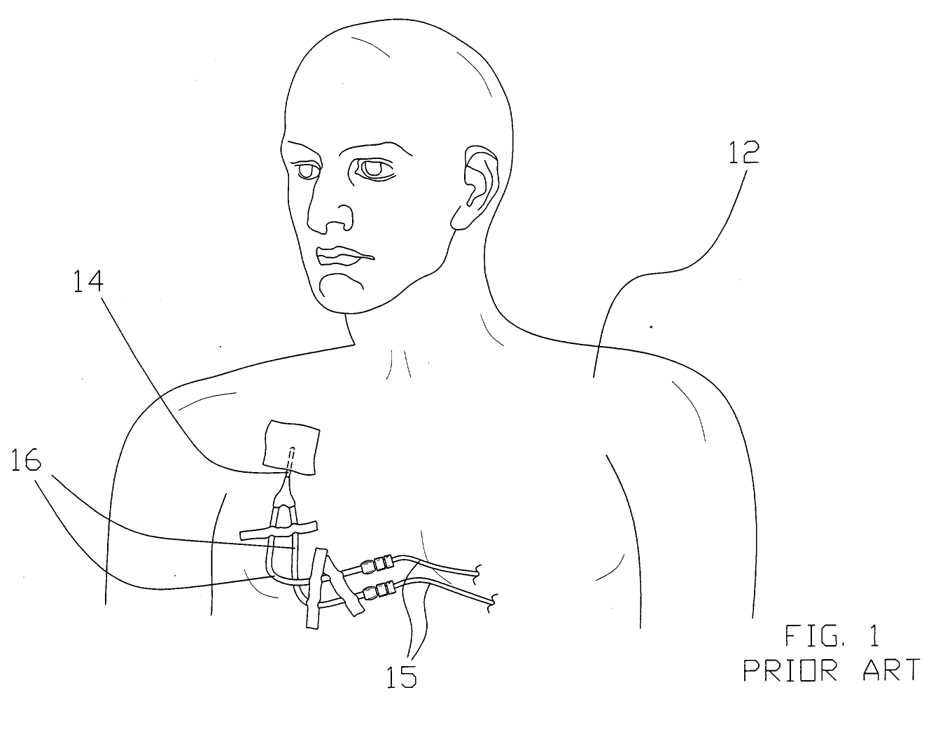

Turning now descriptively to the drawings, in which similar reference characters denote similar elements throughout the several views, FIGS. 1 through 5 illustrate a catheter support system 10, which comprises a support loop 20 positionable about the neck of a patient 12, a connector member 30 for fluidly connecting the catheter tubes 16 and supply tubes 15, and an eyelet member 32 attached to the connector member 30 for receiving the support loop 20. If a force is applied to either the supply tubes 15 or the distal end of the catheter tubes 16, the support loop 20 is pulled against the neck of the patient 12 for both supporting the catheter tube 16 and warning the patient 12 of the force being applied thereto.

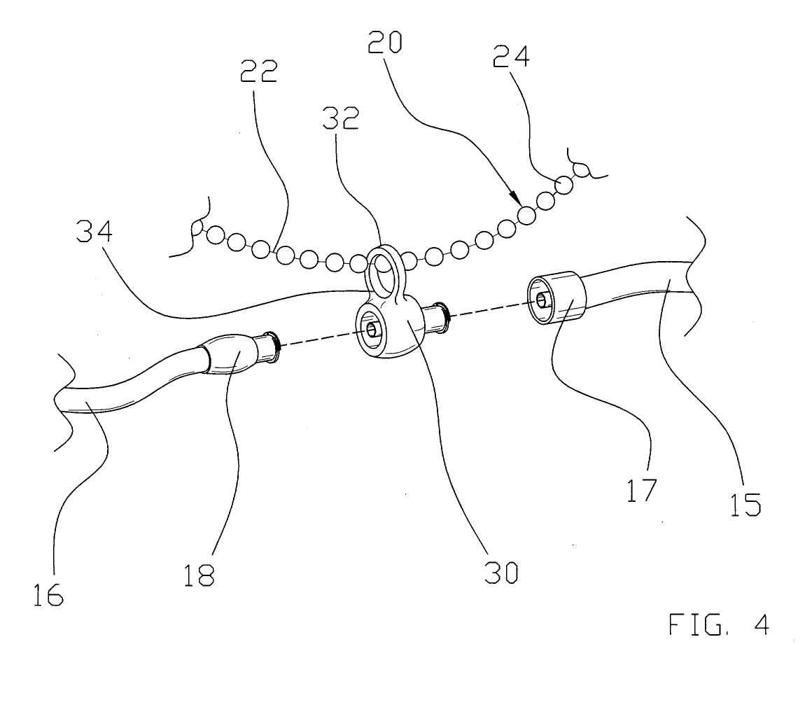

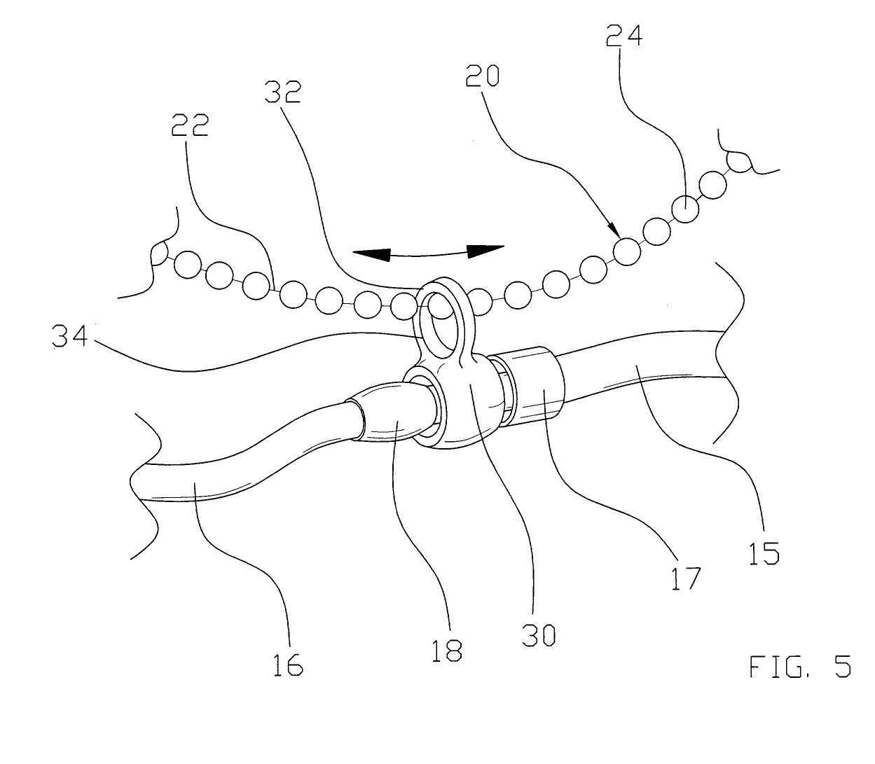

FIG. 4 best illustrates the connector member 30 which is capable of fluidly connecting a catheter tube 16 with a supply tube 15 or end cap (not shown). The connector member 30 has two opposing open ends that are connectable with a first connector 17 and a second connector 18 of the respective catheter tubes 16 and supply tubes 15 as illustrated in FIGS. 4 and 5 of the drawings. A cap member (not shown) may be attached to the connector member 30 which is commonly utilized in the medical industry. Devices in the medical industry are commonly utilized that allow for the connection of a catheter tube 16 and a supply tube 15 which may be utilized to construct the connector member 30. The connector member 30 is preferably comprised of a plastic material, however various other materials may be utilized to construct the connector member 30.

FIGS. 4 and 5 further illustrate an eyelet member 32 attached to the connector member 30. The eyelet member 32 is a looped structure that has an opening within for receiving the support loop 20. The opening within the eyelet member 32 is preferably aligned with the opposing open ends of the connector member 30 as further shown in FIGS. 4 and 5 of the drawings. The eyelet member 32 is preferably comprised of a plastic material, however various other materials may be utilized to construct the eyelet member 32.

It can be appreciated that the eyelet member 32 may be rotated with respect to the connector member 30 to be positioned at various different angles. In addition, the eyelet member 32 may be movably attached to the connector member 30, though the eyelet member 32 is preferably non-movably attached to the connector member 30 as illustrated in FIGS. 4 and 5 of the drawings.

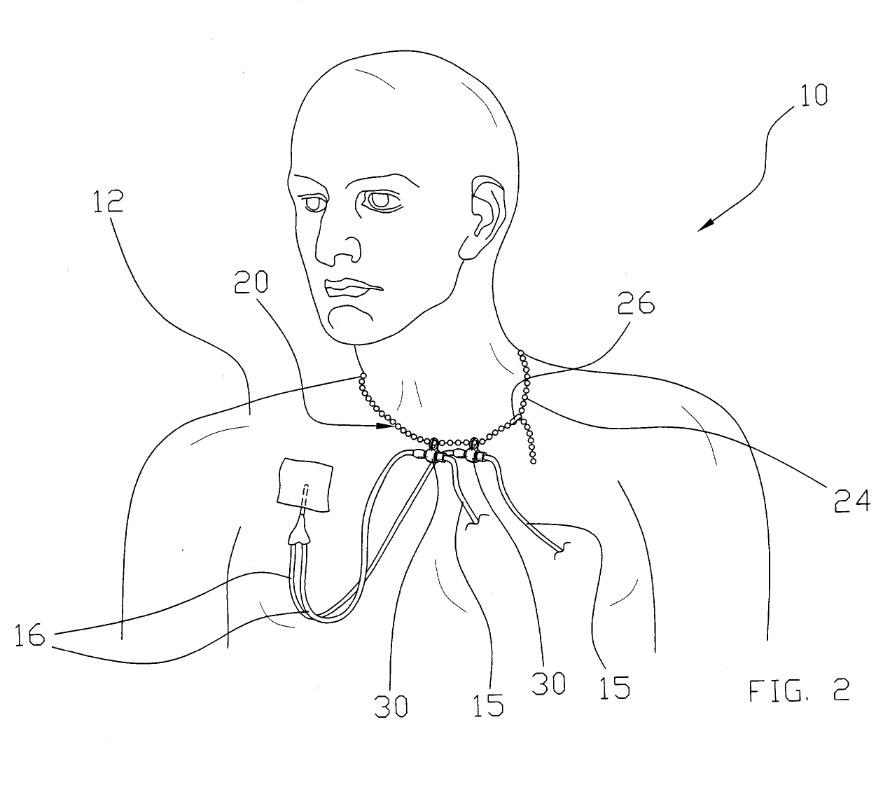

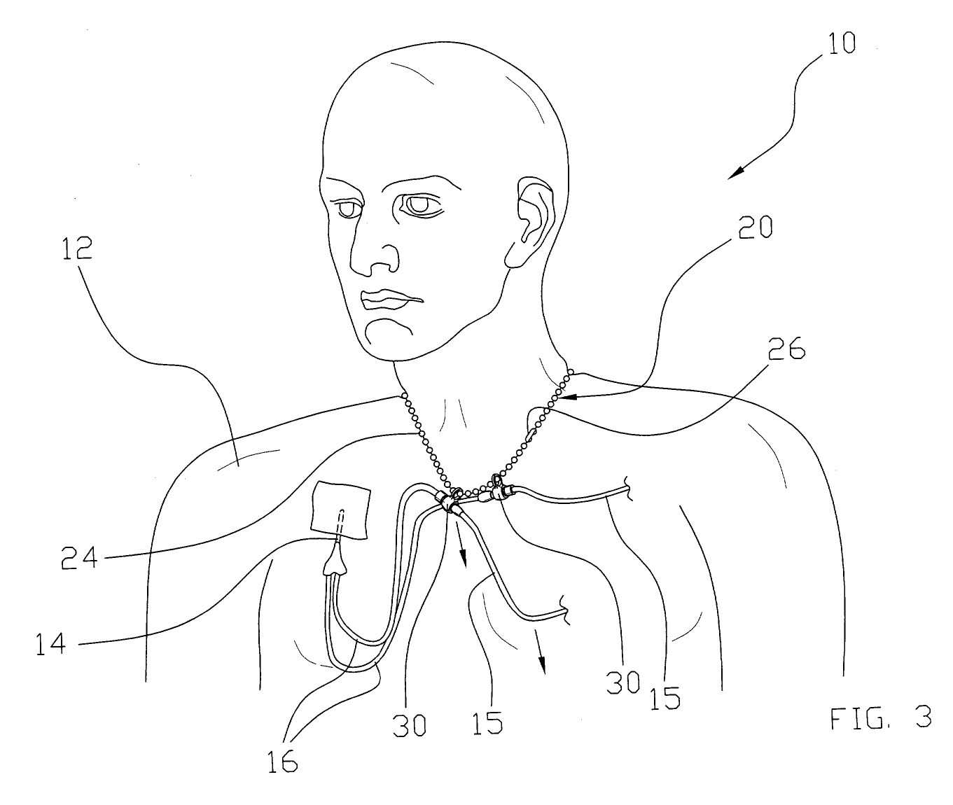

As shown in FIGS. 2 and 3 of the drawings, a support loop 20 is provided that is positionable about the neck of the patient 12. The support loop 20 may be comprised of various materials and structures commonly utilized in the necklace industry. FIGS. 2 through 5 illustrate an exemplary support loop 20 comprised of an elongate band member 22 with a plurality of bead members 24 attached to the band member 22, and an adjustable connector 26 attached to a distal end of the support loop 20 for adjustably connecting to various locations upon the support loop 20.

The band member 22 may be comprised of more than one segment forming a single elongate flexible structure when connected together. The bead members 24 may be comprised of a translucent nylon material or similar material. The adjustable connector 26 may be comprised of a slit-type clasp structure. Various other structures may be utilized to construct the support loop 20.

In use, the user first inserts the support loop 20 through at least one eyelet member 32. If more than one catheter tube 16 is to be utilized, then more than one eyelet member 32 will be attached upon the support loop 20. The user positions the support loop 20 about the neck of the patient 12. The support loop 20 is adjusted using the adjustable connector 26 to achieve the desired fit about the neck of the patient 12. The first connector 17 and a second connector 18 of opposing ends of the supply tube 15 and the catheter tube 16 are thereafter connected together by connecting to the connector member 30. The eyelet member 32 is formed to allow for sliding upon the support loop 20 to allow for increased comfort to the patient 12. If a force is applied a the connector member 30 or either the distal end of the catheter tube 16 or on the supply tube 15 as shown in FIG. 3, the support loop 20 becomes tensioned and provides a tactile warning to the patient 12 that an undesirable force is being applied to the catheter tube 16.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed to be within the expertise of those skilled in the art, and all equivalent structural variations and relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A catheter support system, comprising:
a support loop positionable about a neck of a patient;
a connector member; and
an eyelet member having an opening, wherein said eyelet member extends from said connector member for receiving said support loop.

2. The catheter support system of claim 1, wherein said opening of said eyelet member is aligned with opposing ends of said connector member.

3. The catheter support system of claim 2, wherein said eyelet member has a loop structure.

4. The catheter support system of claim 1, wherein said eyelet member has a loop structure.

5. The catheter support system of claim 1, wherein said opening within said eyelet member is sufficient in size for slidably receiving said support loop.

6. The catheter support system of claim 1, wherein said support loop is adjustable.

7. The catheter support system of claim 6, wherein said support loop is comprised of an elongate band member with a plurality of bead members, and an adjustable connector attached to a distal end of said support loop for selectively engaging one of said plurality of bead members.

8. The catheter support system of claim 1, wherein said connector member fluidly connects a catheter tube to a supply tube or an end cap.

9. The catheter support system of claim 1, wherein said eyelet member is traverse with respect to a longitudinal axis of said connector member.

10. The catheter support system of claim 1, wherein said support loop has two opposing ends connectable to one another by a connector member.

11. A method of utilizing a catheter support system, said method comprising the steps of:
(a) securing a catheter tube to said connector member;
(b) securing a supply tube or an end cap to said connector member opposite of said catheter tube;
(c) positioning an eyelet member of a connector member upon a support loop; and
(d) positioning said support loop about a neck of a patient.

12. The method of utilizing a catheter support system of claim 11, wherein said opening of said eyelet member is aligned with opposing ends of said connector member.

13. The method of utilizing a catheter support system of claim 12, wherein said eyelet member has a loop structure.

14. The method of utilizing a catheter support system of claim 11, wherein said eyelet member has a loop structure.

15. The method of utilizing a catheter support system of claim 11, wherein said opening within said eyelet member is sufficient in size for slidably receiving said support loop.

16. The method of utilizing a catheter support system of claim 11, wherein said support loop is adjustable.

17. The method of utilizing a catheter support system of claim 16, wherein said support loop is comprised of an elongate band member with a plurality of bead members, and an adjustable connector attached to a distal end of said support loop for selectively engaging one of said plurality of bead members.

18. The method of utilizing a catheter support system of claim 11, wherein said connector member fluidly connects a catheter tube to a supply tube.

19. The method of utilizing a catheter support system of claim 11, wherein said eyelet member is traverse with respect to a longitudinal axis of said connector member.

20. A method of utilizing a catheter support system, said method comprising the steps of:
(a) securing a catheter tube to said connector member;
(b) securing a supply tube or an end cap to said connector member opposite of said catheter tube;
(c) positioning an eyelet member of a connector member upon a support loop;
(d) positioning said support loop about a neck of a patient; and
(e) securing said support loop to a desired length about said neck of said patient.
